SEQUENCE LISTING (29 sequences)

SEQ ID NO: 1 - protein, 64 aa, Artificial Sequence
  Description of Artificial Sequence Human Immunodeficiency Virus
RIKQIINMWQKVGKAMYARKSINIGPGRAFSLKPCVKTPLCVRKSIQIGPGRAFSCNTSVITQA

SEQ ID NO: 2 - protein, 64 aa, Artificial Sequence
  Description of Artificial Sequence Human Immunodeficiency Virus
RIKQIINMWQKVGKAMYARKSIHIGPGRAFSLKPCVKTPLCVRKSIHIAPGRAFSCNTSVITQA

SEQ ID NO: 3 - protein, 63 aa, Artificial Sequence
  Description of Artificial Sequence Human Immunodeficiency Virus
IKQIINMWQKVGKAMYARKSIHIGPGRAFSLKPCVKTPLCVRRRISMGPGRVLSCNTSVITQA

SEQ ID NO: 4 - protein, 64 aa, Artificial Sequence
  Description of Artificial Sequence Human Immunodeficiency Virus
RIKQIINMWQKVGKAMYARKRISLGPGRVYSLKPCVKTPLCVRKRMTLGPGKVFSCNTSVITQA

SEQ ID NO: 5 - protein, 64 aa, Artificial Sequence
  Description of Artificial Sequence Human Immunodeficiency Virus
RIKQIINMWQKVGKAMYAQRIIHIGPGRPFSLKPCVKTPLCVRIRIHRGYGRSFSCNTSVITQA

SEQ ID NO: 6 - protein, 64 aa, Artificial Sequence
  Description of Artificial Sequence Human Immunodeficiency Virus
RIKQIINMWQKVGKAMYARGSIHLHPGRKFSLKPCVKTPLCVRKSINMGPGRAFSCNTSVITQA

SEQ ID NO: 7 - protein, 64 aa, Artificial Sequence
  Description of Artificial Sequence Human Immunodeficiency Virus
RIKQIINMWQKVGKAMYARKSIRIGPGQTFSLKPCVKTPLCVRKSVRIGPGQTFSCNTSVITQA

SEQ ID NO: 8 - protein, 64 aa, Artificial Sequence
  Description of Artificial Sequence Human Immunodeficiency Virus
RIKQIINMWQKVGKAMYARESIRIGPGQTFSLKPCVKTPLCVRRSIRIGPGQAFSCNTSVITQA

SEQ ID NO: 9 - protein, 64 aa, Artificial Sequence
  Description of Artificial Sequence Human Immunodeficiency Virus
RIKQIINMWQKVGKAMYARKGIRIGPGQTFSLKPCVKTPLCVRKSIRIGPGQAFSCNTSVITQA

SEQ ID NO: 10 - protein, 64 aa, Artificial Sequence
  Description of Artificial Sequence Human Immunodeficiency Virus
RIKQIINMWQKVGKAMYARKSMRIGPGQTFSLKPCVKTPLCVRKSIRIGPGQTLSCNTSVITQA

SEQ ID NO: 11 - protein, 64 aa, Artificial Sequence
  Description of Artificial Sequence Human Immunodeficiency Virus
RIKQIINMWQKVGKAMYARKSVRIGPGQTSSLKPCVKTPLCVRRSIRIGPGQTFSCNTSVITQA

SEQ ID NO: 12 - protein, 64 aa, Artificial Sequence
  Description of Artificial Sequence Human Immunodeficiency Virus
RIKQIINMWQKVGKAMYARQSIRIGPGQAFSLKPCVKTPLCVRKSVRIGPGQAFSCNTSVITQA

SEQ ID NO: 13 - protein, 64 aa, Artificial Sequence
  Description of Artificial Sequence Human Immunodeficiency Virus
RIKQIINMWQKVGKAMYARKGIHIGPGQAFSLKPCVKTPLCVRKGIGIGPGQTFSCNTSVITQA

SEQ ID NO: 14 - protein, 64 aa, Artificial Sequence
  Description of Artificial Sequence Human Immunodeficiency Virus
RIKQIINMWQKVGKAMYARESIGIGPGQAFSLKPCVKTPLCVRKSMRIGPGQTFSCNTSVITQA

SEQ ID NO: 15 - protein, 46 aa, Artificial Sequence
  Description of Artificial Sequence Human Immunodeficiency Virus
RKSIHIGPGRAFSLKPCVKTPLCVRKSIHIAPGRAFSCNTSVITQA

SEQ ID NO: 16 - protein, 52 aa, Artificial Sequence
  Description of Artificial Sequence Human Immunodeficiency Virus
RIKQIINMWQKVGKAMYARKSIHIGPGRAFRKSIHIAPGRAFSCNTSVITQA

SEQ ID NO: 17 - protein, 42 aa, Artificial Sequence
  Description of Artificial Sequence Human Immunodeficiency Virus
RIKQIINMWQKVGKAMYARKSIHIGPGRAFRKSIHIAPGRAF

SEQ ID NO: 18 - protein, 54 aa, Artificial Sequence
  Description of Artificial Sequence Human Immunodeficiency Virus
RIKQIINMWQKVGKAMYARKSIHIGPGRAFSLKPCVKTPLCVRKSIHIAPGRAF

SEQ ID NO: 19 - protein, 36 aa, Artificial Sequence
  Description of Artificial Sequence Human Immunodeficiency Virus
RKSIHIGPGRAFSLKPCVKTPLCVRKSIHIAPGRAF

SEQ ID NO: 20 - protein, 56 aa, Artificial Sequence
  Description of Artificial Sequence Human Immunodeficiency Virus
RIKQIINMWQKVGKAMYASIGPGRAFSLKPCVKTPLCVSIGPGRAFSCNTSVITQA

SEQ ID NO: 21 - protein, 64 aa, Artificial Sequence
  Description of Artificial Sequence Human Immunodeficiency Virus
RIKQIINMWQKVGKAMYARRNIHIGLGRRFSLKPCVKTPLCVRRSVRIGPGGAMSCNTSVITQA

SEQ ID NO: 22 - protein, 64 aa, Artificial Sequence
  Description of Artificial Sequence Human Immunodeficiency Virus
RIKQIINMWQKVGKAMYARRSIPIGPGRAFSLKPCVKTPLCVVRKIPIGPGSSFSCNTSVITQA

SEQ ID NO: 23 - protein, 64 aa, Artificial Sequence
  Description of Artificial Sequence Human Immunodeficiency Virus
RIKQIINMWQKVGKAMYARKRIPLGLGKAFSLKPCVKTPLCVRKGIHLGPGRAISCNTSVITQA

SEQ ID NO: 24 - protein, 64 aa, Artificial Sequence
  Description of Artificial Sequence Human Immunodeficiency Virus
RIKQIINMWQKVGKAMYARKGIHMGPGKAISLKPCVKTPLCVRRGIPIGPGRAFSCNTSVITQA

SEQ ID NO: 25 - protein, 64 aa, Artificial Sequence
  Description of Artificial Sequence Human Immunodeficiency Virus
RIKQIINMWQKVGKAMYARKSIRIGPGRAVSLKPCVKTPLCVRRRISIGPGRAFSCNTSVITQA

SEQ ID NO: 26 - protein, 64 aa, Artificial Sequence
  Description of Artificial Sequence Human Immunodeficiency Virus
RIKQIINMWQKVGKAMYARRSIHLGMGRALSLKPCVKTPLCVRRSIHMGLGRAFSCNTSVITQA

SEQ ID NO: 27 - protein, 64 aa, Artificial Sequence
  Description of Artificial Sequence Human Immunodeficiency Virus
RIKQIINMWQKVGKAMYARKGINIGPGRAFSLKPCVKTPLCVRKGIHIGPGRTFSCNTSVITQA

SEQ ID NO: 28 - protein, 64 aa, Artificial Sequence
  Description of Artificial Sequence Human Immunodeficiency Virus
RIKQIINMWQKVGKAMYAIRIGHIGPGRAFSLKPCVKTPLCVRRHIHIGPGRAFSCNTSVITQA

SEQ ID NO: 29 - protein, 66 aa, Artificial Sequence
  Description of Artificial Sequence Human Immunodeficiency Virus
RIKQIINMWQKVGKAMYARRKGIHIGPGRAISLKPCVKTPLCVTGKSIRMGLGRAWSCNTSVITQA

(19) United States
(12) Patent Application Publication
(10) Pub. No.: US 2003/0219452 A1
(43) Pub. Date: Nov. 27, 2003

(54) HIV envelope V3-CCR5 binding site immunogen

(76) Inventors: Barton F. Haynes, Durham, NC (US); Bette T. Korber, Los Alamos, NM (US)

Correspondence Address: NIXON & VANDERHYE, PC, 1100 N GLEBE ROAD, 8TH FLOOR, ARLINGTON, VA, 22201-4714, US

(21) Appl. No.: 10/305,357

(22) Filed: Nov. 27, 2002

Related U.S. Application Data
(60) Provisional application No. 60/333,148, filed on Nov. 27, 2001.

(51) Int. Cl. A61K 39/21; C07K 14/16

(52) U.S. Cl. 424/188.1; 530/350

(57) ABSTRACT

The present invention relates, in general, to an immunogen and, in particular, to an immunogen for inducing antibodies that neutralize a wide spectrum of HIV primary isolates. The invention also relates to a method of inducing anti-HIV antibodies using same.

This application claims priority from U.S. Provisional Application No. 60/333,148, filed Nov. 27, 2001, the entire content of which is incorporated herein by reference.

### TECHNICAL FIELD

The present invention relates, in general, to an immunogen and, in particular, to an immunogen for inducing antibodies that neutralize a wide spectrum of HIV primary isolates. The invention also relates to a method of inducing anti-HIV antibodies using such an immunogen.

### BACKGROUND

As the HIV epidemic continues to spread world-wide, the need for an effective HIV vaccine remains urgent. A key obstacle to HIV vaccine development is the extraordinary variability of HIV and the rapidity and extent of HIV mutation (Wain-Hobson in The Evolutionary biology of Retroviruses, SSB Morse Ed. Raven Press, NY, pgs 185-209 (1994))

Myers, Korber and colleagues have analyzed HIV sequences worldwide and divided HIV isolates into groups or clades, and provided a basis for evaluating the evolutionary relationship of individual HIV isolates to each other (Myers et al (Eds), Human Retroviruses and AIDS (1995), Published by Theoretical Biology and Biophysics Group, T-10, Mail Stop K710, Los Alamos National Laboratory, Los Alamos, N. Mex. 87545). The degree of variation in HIV protein regions that contain CTL and T helper epitopes has also recently been analyzed by Korber et al, and sequence variation documented in many CTL and T helper epitopes among HIV isolates (Korber et al (Eds), HIV Molecular Immunology Database (1995), Published by Theoretical Biology and Biophysics Group, Los Alamos National Laboratory, Los Alamos, N. Mex. 87545).

A new level of HIV variation complexity was recently reported by Hahn et al by demonstrating the frequent recombination of HIV among clades (Robinson et al, J. Mol. Evol. 40:245-259 (1995)). These authors suggest that as many as 10% of HIV isolates are mosaics of recombination, suggesting that vaccines based on only one HIV clade will not protect immunized subjects from mosaic HIV isolates (Robinson et al, J. Mol. Evol 40:245-259 (1995)).

The present invention relates to an immunogen suitable for use in an HIV vaccine. The immunogen will induce broadly cross-reactive neutralizing antibodies in humans and neutralize a wide spectrum of HIV primary isolates.

### SUMMARY OF THE INVENTION

The present invention relates to an immunogen for inducing antibodies that neutralize a wide spectrum of HIV primary isolates. The invention also relates to a method of inducing anti-HIV antibodies using such an immunogen.

Objects and advantages of the present invention will be clear from the description that follows.

### BRIEF DESCRIPTION OF THE DRAWINGS

FIG. 1. Peptide immunogen design.

FIG. 2. Sequence of CBLH-1-89.6P.

### DETAILED DESCRIPTION OF THE INVENTION

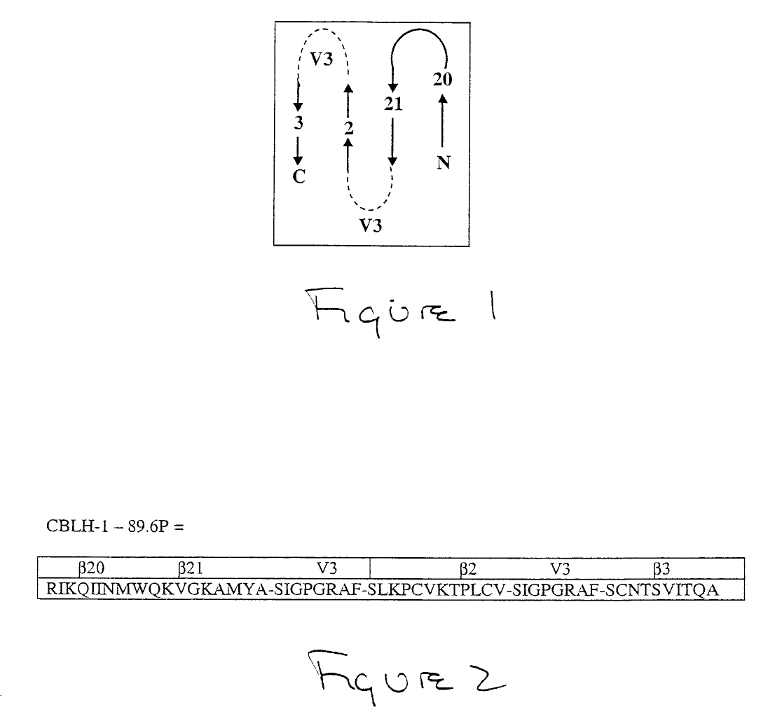

Targets that induce antibodies that neutralize primary isolates of HIV include the gp120V3 loop and the CCR5 (cellular HIV co-receptor) binding site. Kwong et al (Nature 393:648-659 (1968)) have shown that the CCR5 binding site is adjacent to the base of the V3 loop and is formed by the juxtaposition of 4 anti-parallel beta-pleated sheets. The present invention provides a peptide immunogen that induces antibodies that neutralize HIV primary isolates comprising components of both the HIV gp120 CCR5 binding site and the V3 loop. The immunogen has the design set forth in FIG. 1.

The peptide immunogen of the invention, designated CCR5 binding site/V3, with the CBLH-1 peptide being the prototype, comprises, from the N-terminus to the C-terminus, beta sheet regions 20, 21, 2 and 3 (see Nature 393:650 (1998)). A V3 loop sequence connects beta sheets 21 and 2 and a V3 loop sequence is present between beta sheets 2 and 3, which site is naturally occupied by the V1-V2 loops. Accordingly, the peptide immunogen of the invention comprises 4 anti-paralleled beta sheet sequences that reflect the CCR5 binding site and 2 V3 loops. The V3 loops can vary in length (for example, from about 8 to about 16 amino acids). In a preferred embodiment, the 4 beta sheets correspond to disparate gp120 regions. In CCR5 binding site/V3, CBLH-1, they are present in a linear peptide comprising V3 loops.

A multiplicity of peptide immunogens of the present invention can be formulated as a composition suitable for administration as a vaccine. The V3 components of the peptide immunogens of the invention present in the instant composition are selected so as to be representative of higher order structural motifs present in a population, which motifs mediate V3 functions in the course of envelope mediated HIV interaction with host cells. The Los Alamos National Laboratories Human Retroviruses and AIDS Database (Human Retroviruses and AIDS, 2000, Published by the Theoretical Biology and Biophysics G T-10, Mail Stop K710, LANL, Los Alamos, N. Mex.) presently contains over 14,000 HIV V3 envelope sequences, showing the extraordinary diversity the virus has obtained since originating in man in Africa approximately 50 years ago. For example, among 432 HIV-1 V3 sequences derived from individuals infected with subtype C (designated “Clade C”) in Africa currently available in the HIV database, 176 distinct variants of a 23 amino acid stretch at the tip of the V3 loop have been found. Similarly, among 6870 B subtype (designated “Clade B”) V3 sequences from the US, 1514 unique forms have been found.

A method has been developed to organize short antigenic domains by protein similarity scores using maximum-linkage clustering. This method enables the visualization of the clustering patterns as a dendrogram, and the splitting patterns in the dendrogram can be used to define clusters of related sequences (Korber et al, J. Virol. 68:6730-6744 (1994)). The method allows the use of several different amino acid similarity scoring schemes available in the literature, preferred is the amino acid substitution matrix developed by Henikoff and Henikoff (see Advances in Protein Chemistry 54:73-97 (2000) and Proteins: Structure, Function and Genetics 17:49-61 (1993)), designed to give substitutions that are well tolerated in conserved protein structural elements a high score, and a low score to those that are not. Typically excluded from consideration very rare, highly divergent peptides, and favored are peptides found in many individuals within the population. In a selected set of sequences, most of the unique forms are within one or two amino acids from a least one other of the peptides chosen. This method has been applied to clustering the large number of variants of the antigenic tip of the V3 domain within Clade B and Clade C into groups (about 25) that are likely to be cross-reactive within the group. Based on these clustering patterns, variants (e.g., about 25-30) are selected that are representative or “central” to each group, for testing for antigenicity. The HIV Clade B and Clade C gp120 envelope V3 sequences have been analyzed, as described above, for groups of V3 sequences predicted to have structural similarities. Twenty five Clade C and 30 Clade B groups have been defined, and chosen out of each group is a common, or the most common, sequence as a representative of that group.

Shown in Tables 3 and 4 are examples of immunogens of the present invention for HIV Clades B and C, respectively. The immunogens of B can be combined to provide a composition suitable for use in the US (clade B) and Africa (Clade C).

TABLE 3

396.2/170.6-RIKQIINMWQKVGKAMYA-RRNIHIGLGRRF-
SLKPCVKTPLCV-RRSVRIGPGGAM-SCNTSVITQA
82.15/144.8-RIKQIINMWQKVGKAMYA-RRSIPIGPGRAF-
SLKPCVKTPLCV-VRKIPIGPGSSF-SCNTSVITQA
23.38/365.2-RIKQIINMWQKVGKAMYA-RKRIPLGLGKAF-
SLKPCVKTPLCV-RKGIHLGPGPAI-SCNTSVITQA
513.2/1448.1-RIKQIINMWQKVGKAMYA-RKGIHMGPGKAI-
SLKPCVKTPLCV-RRGIPIGPGRAF-SCNTSVITQA
69.18/146.8-RIKQIINMWQKVGKAMYA-RKSIRIGPGRAV-
SLKPCVKTPLCV-RRRISIGPGRAF-SCNTSVITQA
113.10/51.23-RIKQIINMWQKVGKAMYA-RRSIHLGMGPAL-
SLKPCVKTPLCV-RRSIHMGLGRAF-SCNTSVITQA
72.18/36.29-RIKQIINMWQKVGKAMYA-RKGINIGPGRAF-
SLKPCVKTPLCV-RKGIHIGPGRTF-SCNTSVITQA
70.18/89.14-RIKQIINMWQKVGKAMYA-IRIGHIGPGRAF-
SLKPCVKTPLCV- RRHIHIGPGRAF-SCNTSVITQA
163.7/57.20-RIKQIINMWQKVGKAMYA-RRKGIHIGPGRAI-
SLKPCVKTPLCV-TGKSIRMGLGRAW-SCNTSVITQA
11.85/34.29-RIKQIINMWQKVGKAMYA-RKSINIGPGRAF-
SLKPCVKTPLCV-RKSIQIGPGPAF-SCNTSVITQA
1.481/85.15-RIKQIINMWQKVGKAMYA-RKSIHIGPGRAF-
SLKPCVKTPLCV-RKSIHIAPGRAF-SCNTSVITQA
62.19/125.9-IKQIINMWQKVGKAMYA-RKSIHIGPGRAF-
SLKPCVKTPLCV-RRRISMGPGRVL-SCNTSVITQA
35.29/74.17-RIKQIINMWQKVGKAMYA-RKRISLGPGRVY-
SLKPCVKTPLCV-RKRMTLGPGKVF-SCNTSVITQA
46.26/122.9-RIKQIINMWQKVGKAMYA-QRIIHIGPGRPF-
SLKPCVKTPLCV-RIRIHRGYGRSF-SCNTSVITQA
162.7/3.323-RIKQIINMWQKVGKAMYA-RGSIHLHPGRKF-
SLKPCVKTPLCV-RKSINMGPGRAF-SCNTSVITQA

TABLE 4
1(4)-RIKQIINMWQKVGKAMYA-rksirigpGqtf-SLKPCVKTPLCV-
rksVrigpGqtf-SCNTSVITQA
7(8)-RIKQIINMWQKVGKAMYA-rEsirigpGqtf-SLKPCVKTPLCV-
rRsorogpGqAF-SCNTSVITQA
9(10)-RIKQIINMWQKVGKAMYA-rkGirigpGqtf-SLKPCVKTPLCV-
rksirigpGqAF-SCNTSVITQA
14(15)-RIKQIINMWQKVGKAMYA-rksMrigpGqtf-SLKPCVKTPLCV-
rksirigpGqtL-SCNTSVITQA
16(17)-RIKQIINMWQKVGKAMYA-rksVrigpGqtS-SLKPCVKTPLCV-
rRsirigpGqtf-SCNTSVITQA
20(22)-RIKQIINMWQKVGKAMYA-rQsirigpGqAF-SLKPCVKTPLCV-
rksVrigpGqAF-SCNTSVITQA
23(24)-RIKQIINMWQKVGKAMYA-rkGiHigpGqAf-SLKPCVKTPLCV-
rkGiGigpGqtf-SCNTSVITQA
25(14)-RIKQIINMWQKVGKAMYA-rEsiGigpGqAf-SLKPCVKTPLCV-
rksMrigpGqtf-SCNTSVITQA

While the above is offered by way of example, it will be appreciated that the same analyses can by performed for HIV Clades A, D, E, F, G, H, M, N, O, etc, to design immunogens that react with HIV primary isolates from these Clades. The length of the V3 inserts in the present immunogens can vary, for example, from about 8 to about 16 amino acids. In a similar manner, analysis can be made of amino acid heterogeneity with the 2, 3, 20 and 21 beta sheet regions of gp120 and multiple HIV (chemokine) receptor binding site sequences can be used in peptide design.

The peptide immunogens of the invention can be chemically synthesized and purified using methods which are well known to the ordinarily skilled artisan. The composition can comprise the peptides linked end to end or can comprise a mixture of individual peptides. The peptide immunogens can also be synthesized by well-known recombinant DNA techniques. Recombinant synthesis may be preferred when the peptides are covalently linked.

Nucleic acids encoding the peptides of the invention can be used as components of a DNA vaccine wherein the peptide encoding sequence(s) is/are administered as naked DNA or, for example, a minigene encoding the peptides can be present in a viral vector, such as an adenoviral vector, a modified vaccinia ankara vector, a vaccinia vector or an attenuated TB vector. Expression of the immunogenic peptides of the invention can be induced in a patient's own cells, by introduction into those cells of nucleic acids that encode the peptides, preferably using codons and promoters that optimize expression in human cells. Examples of methods of making and using DNA vaccines are disclosed in U.S. Pat. Nos. 5,580,859, 5,589,466, and 5,703,055.

The composition of the invention comprises an immunologically effective amount of the peptide immunogens of this invention, or DNA sequence(s) encoding same, in a pharmaceutically acceptable delivery system. The compositions can be used for prevention and/or treatment of immunodeficiency virus infection. The compositions of the invention can be formulated using adjuvants, emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosal immunity (e.g, the formulation can be designed for intranasal administration). The present compositions can be administered by any convenient route including subcutaneous, intranasal, oral, intramuscular, or other parenteral or enteral route. The immunogens can be administered as a single dose or multiple doses. Optimum immunization schedules can be readily determined by the ordinarily skilled artisan and can vary with the patient, the composition and the effect sought. By way of example, it is noted that approximately 50 μg-100 μg of each hybrid peptide can be administered, for example, intramuscularly (e.g. 3×).

The invention contemplates the direct use of both the peptides of the invention and nucleic acids encoding same. For example, a minigene encoding the peptides can be used as a prime and/or boost.

In addition to the composition described above, the invention encompasses each of the immunogens disclosed as well as each of the components (V3 and CCR5), alone or in covalent or non-covalent association with other sequences. The invention further encompasses nucleic acid sequences encoding any and all such peptides.

Certain aspects of the invention are described in greater detail in the non-limiting Example that follows.

### EXAMPLE

A peptide immunogen of the invention, designated CBLH-1-89.6P) and having the sequence shown in FIG. 2 was tested for both immunogenicity with antibodies against the peptide and for neutralizing antibodies. Shown in Table 1 are the results of immunization of guinea pigs twice with CBLH-1 of SHIV89.6P in complete Freund's adjuvant (CFA)/incomplete Freund's adjuvant (IFA) versus immunization of guinea pigs twice with another immunogen, the C4-V3 gp120 immunogen (see Provisional Application No. 60/331,036).

TABLE 1

| | | Titer to Immunizing |
|---|---|---|
| Animal | | peptide after |
| number | Immunogen | 2 Immunizations |
| 322 | CBLH-1 of SHIV89.6P | 102,400 |
| 323 | CBLH-1 of SHIV 89.6P | 204,800 |
| 324 | CBLH-1 of SHIV 89.6P | 102,400 |
| 325 | C4-V3 89.6P | 25,600 |
| 326 | C4-V3 89.6P | 12,800 |
| 327 | C4-V3 89.6P | 12,800 |

Table 2 shows the neutralizing antibody results of the sera of the same animals against several HIV primary isolates.

TABLE 2

| | | | Nab titer | | % p24 reduction | |
|---|---|---|---|---|---|---|
| | Immun- | | in MT-2 cells¹ | | in PBMC² | |
| Animal | ogen | Bleed | HIV-1MN | SHIV-89.6P | SF162 | JR-FL |
| 322 | CBLH-1 | Pre | 0 | 0 | 0 | 0 |
| | | 1 | 0 | 0 | 0 | 0 |
| | | 2 | 0 | 0 | 0 | 0 |
| | | 3 | 122 | 0 | 0 | 0 |
| | | 4 | 75 | 0 | 0 | 0 |
| 323 | CBLH-1 | Pre | 0 | 0 | 0 | 0 |
| | | 1 | 42 | 0 | 0 | 0 |
| | | 2 | 444 | 0 | 0 | 0 |
| | | 3 | >540 | 0 | 100 | 88 |
| | | 4 | >540 | 0 | 100 | 0 |
| 324 | CBLH-1 | Pre | 0 | 0 | 0 | 0 |
| | | 1 | 0 | 0 | 0 | 0 |
| | | 2 | 188 | 0 | 0 | 0 |
| | | 3 | >540 | 0 | 89 | 0 |
| | | 4 | >540 | 24 | 93 | 0 |
| 325 | C4-V3 | Pre | 0 | 0 | 0 | 0 |
| | 89.6P | 1 | 0 | 0 | 0 | 0 |
| | | 2 | 0 | 0 | 0 | 0 |
| | | 3 | 0 | 0 | 0 | 0 |
| | | 4 | 0 | 23 | 0 | 0 |
| 326 | C4-V3 | Pre | 0 | 0 | 0 | 0 |
| | 89.6P | 1 | 79 | 0 | 0 | 0 |
| | | 2 | 131 | 0 | 0 | 0 |
| | | 3 | 53 | 0 | 0 | 0 |
| | | 4 | 47 | 0 | 0 | 0 |
| 327 | C4-V3 | Pre | 0 | 0 | 0 | 0 |
| | 89.6P | 1 | 81 | 0 | 0 | 0 |
| | | 2 | | 0 | 0 | 0 |
| | | 3 | | 0 | 0 | 0 |
| | | 4 | | 0 | 0 | 0 |

¹NAb titers are the reciprocal serum dilution at which 50% of cells were protected from virus-induced killing as measured by neutral red uptake.
²Samples were assayed at a 1:4 dilution in triplicate. % reduction in p24 is calculated relative to the amount of p24 produced in the presence of the corresponding prebleed sample.

The results shown in Table 2 demonstrate that whereas C4-V3 neutralization titers were low and did not cross neutralize any HIV primary isolates, CBLH-1 of SHIV89.6P immunization of animals 323 and 342 induced antibodies that cross-neutralized HIV SF162 and animal 323 also cross-neutralized the primary isolate HTV JR-FL.

The following peptides have also been designed and may represent immunogenic truncated variants of CCR5 binding site/V3 peptide constructs:

1.481/85.15-Delta 20/21-RKSIHIGPGRAF-SLKPCVKTPLCV -RKSIHIAPGRAF-SCNTSVITQA

1.481/85.15-Delta 2-RIKQIINMWQKVGKAMYA-RKSIHIGPGRAF -RKSIHIAPGRAF-SCNTSVTTQA

1.481/85.15-Delta 2/3-RIKQIINMWQKVGKAMYA -RKSIHIGPGRAF-RKSIHIAPGRAF

1.481/85.15-Delta 3-RTKQIINMWQKVGKAMYA-RKSIHIGPGRAF -SLKPCVKTPLCV-RKSIHIAPGRAF

1.481/85.15-Delta 20/21/3-RKSIHIGPGRAF-SLKPCVKTPLCV-RKSIHIAPGRAF.

All documents cited above are hereby incorporated in their entirety by reference.

One skilled in the art will appreciate from a reading of this disclosure that various changes in form and detail can be made without departing from the true scope of the invention.

What is claimed is:

1. A peptide immunogen comprising, from the N-terminus to the C-terminus, beta sheet regions 20, 21, 2 and 3 of a human immunodeficiency virus (HIV) gp120 CCR5 binding site, wherein an HIV gp120 V3 loop sequence is present between said beta sheet regions 21 and 2 and between said beta sheets regions 2 and 3.

2. The peptide according to claim 1 wherein each of said V3 loop sequences comprises from about 8 to about 16 amino acids.

3. The peptide according to claim 1 wherein said beta sheet regions correspond to disparate gp120 regions.

4. A composition comprising at least two peptides according to claim 1.

5. The composition according to claim 4 wherein said at least 2 peptides are covalently linked.

6. A method of inducing an immune response in a patient to HIV comprising administering to said patient at least one peptide according to claim 1 in an amount and under conditions such that said response is induced.

7. A vaccine comprising a multiplicity of peptides according to claim 1 wherein said V3 loop sequences are selected so as to be representative of higher order structural motifs present in a population of HIV isolates.

8. The peptide according to claim 1 wherein said peptide comprises a sequence selected from the group consisting of
RIKQIINMWQKVGKAMYA-RRSIPIGPGRAF-SLKPCVKTPLCV-VRKIPIGPGSSF-SCNTSVITQA;
RIKQIINMWQKVGKAMYA-RKRIPLGLGKAF-SLKPCVKTPLCV-RKGIHLGPGRAI-SCNTSVITQA;
RIKQIINMWQKVGKAMYA-RKGIHMGPGKAI-SLKPCVKTPLCV-RRGIPIGPGRAF-SCNTSVITQA;
RIKQIINMWQKVGKAMYA-RKSIRIGPGRAV-SLKPCVKTPLCV-RRRISIGPGRAF-SCNTSVITQA;
RIKQIINMWQKVGKAMYA-RRSIHLGMGRAL-SLKPCVKTPLCV-RRSIHMGLGRAF-SCNTSVITQA;
RIKQIINMWQKVGKAMYA-RKGINIGPGRAF-SLKPCVKTPLCV-RKGIHIGPGRTF-SCNTSVITQA;
RIKQIINMWQKVGKAMYA-IRIGHIGPGRAF-SLKPCVKTPLCV-RRHIHIGPGRAF-SCNTSVITQA;
RIKQIINMWQKVGKAMYA-RRKGIHIGPGRAI-SLKPCVKTPLCV-TGKSIRMGLGRAW-SCNTSVITQA;
RIKQIINMWQKVGKAMYA-RKSINIGPGRAF-SLKPCVKTPLCV-RKSIQIGPGRAF-SCNTSVITQA;
RIKQIINMWQKVGKAMYA-RKSIHIGPGRAF-SLKPCVKTPLCV-RKSIHIAPGRAF-SCNTSVITQA;
IKQIINMWQKVGKAMYA-RKSIHIGPGRAF-SLKPCVKTPLCV-RRRISMGPGRVL-SCNTSVITQA;
RIKQIINMWQKVGKAMYA-RKRISLGPGRVY-SLKPCVKTPLCV-RKRMTLGPGKVF-SCNTSVITQA;
RIKQIINMWQKVGKAMYA-QRIIHIGPGRPF-SLKPCVKTPLCV-RIRIHRGYGRSF-SCNTSVITQA; and
RIKQIINMWQKVGKAMYA-RGSIHLHPGRKF-SLKPCVKTPLCV-RKSINMGPGRAF-SCNTSVITQA.

9. A composition comprising at least two of said peptides according to claim 8.

10. The peptide according to claim 1 wherein said peptide comprises a sequence selected from the group consisting of
RIKQIINMWQKVGKAMYA-rksirigpGqtf-SLKPCVKTPLCV-rksVrigpGqtf-SCNTSVITQA;
RIKQIINMWQKVGKAMYA-rEsirigpGqtf-SLKPCVKTPLCV-rRsirigpGqAf-SCNTSVITQA;
RIKQIINMWQKVGKAMYA-rkGirigpGqtf-SLKPCVKTPLCV-rksirigpGqAf-SCNTSVITQA;
RIKQIINMWQKVGKAMYA-rksMrigpGqtf-SLKPCVKTPLCV-rksirigpGqtL-SCNTSVITQA;
RIKQIINMWQKVGKAMYA-rksVrigpGqtS-SLKPCVKTPLCV-rRsirigpGqtf-SCNTSVITQA;
RIKQIINMWQKVGKAMYA-rQsirigpGqAf-SLKPCVKTPLCV-rksVrigpGqAf-SCNTSVITQA;
RIKQTINMWQKVGKAMYA-rkGiHigpGqAf-SLKPCVKTPLCV-rkGiGigpGqtf-SCNTSVITQA; and
RIKQIINMWQKVGKAMYA-rEsiGigpGqAf-SLKPCVKTPLCV-rksMrigpGqtf-SCNTSVITQA.

11. A composition comprising at least two of said peptides according to claim 10.

12. A nucleic acid sequence encoding at least one peptide according to claim 1.

13. A composition comprising at least one nucleic acid sequence encoding at least two of said peptides according to claim 1.

14. A method of inducing an immune response in a patient to HIV comprising administering to said patient at least one nucleic acid sequence according to claim 12 under conditions such that said nucleic acid sequence is expressed, said at least one peptide is produced and said immune response is induced.
